(19) United States
(12) Patent Application Publication
(10) Pub. No.: US 2009/0080597 A1
(43) Pub. Date: Mar. 26, 2009

(54) SYSTEM AND METHOD FOR PERFORMING MATERIAL DECOMPOSITION USING AN OVERDETERMINED SYSTEM OF EQUATIONS

(76) Inventor: Samit Kumar Basu, Niskayuna, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/861,639

(22) Filed: Sep. 26, 2007

(51) Int. Cl. A61B 6/03; G06K 9/00

(52) U.S. Cl. 378/5; 382/131

(57) ABSTRACT

A system and method of a diagnostic imaging system includes an x-ray source that emits a beam of x-rays toward an object to be imaged, a detector that receives x-rays emitted by the x-ray source and attenuated by the object, and a data acquisition system (DAS) operably connected to the detector. A computer is operably connected to the DAS and programmed to obtain a number of measurements of energy-sensitive CT measurements in excess of a number of materials to be resolved, decompose the number of measurements into individual materials as an overdetermined system of equations, and generate an image of the individual materials based on the decomposition.

### BACKGROUND OF THE INVENTION

The present invention relates generally to diagnostic imaging and, more particularly, to a system and method of basis material decomposition and representation of diagnostic imaging data at a virtual energy having minimized monochromatic noise or maximized contrast to noise ratio.

Diagnostic devices comprise x-ray systems, magnetic resonance (MR) systems, ultrasound systems, computed tomography (CT) systems, positron emission tomography (PET) systems, ultrasound, nuclear medicine, and other types of imaging systems. Typically, in CT imaging systems, an x-ray source emits a fan-shaped beam toward a subject or object, such as a patient or a piece of luggage. Hereinafter, the terms “subject” and “object” shall include anything capable of being imaged. The beam, after being attenuated by the subject, impinges upon an array of radiation detectors. The intensity of the attenuated beam radiation received at the detector array is typically dependent upon the attenuation of the x-ray beam by the subject. Each detector element of the detector array produces a separate electrical signal indicative of the attenuated beam received by each detector element. The electrical signals are transmitted to a data processing system for analysis which ultimately produces an image.

Generally, the x-ray source and the detector array are rotated about the gantry opening within an imaging plane and around the subject. X-ray sources typically include x-ray tubes, which emit the x-ray beam at a focal point. X-ray detectors typically include a collimator for collimating x-ray beams received at the detector, a scintillator for converting x-rays to light energy adjacent the collimator, and photodiodes for receiving the light energy from the adjacent scintillator and producing electrical signals therefrom.

Typically, each scintillator of a scintillator array converts x-rays to light energy. Each scintillator discharges light energy to a photodiode adjacent thereto. Each photodiode detects the light energy and generates a corresponding electrical signal. The outputs of the photodiodes are then transmitted to the data processing system for image reconstruction.

A CT imaging system may include an energy discriminating (ED), multi energy (ME), and/or dual energy (DE) CT imaging system that may be referred to as an EDCT, MECT, and/or DE-CT imaging system. Such systems may use a direct conversion detector material in lieu of a scintillator. The EDCT, MECT, and/or DE-CT imaging system in an example is configured to be responsive to different x-ray spectra. For example, a conventional third generation CT system may acquire projections sequentially at different peak kilovoltage (kVp) levels, which changes the peak and spectrum of energy of the incident photons comprising the emitted x-ray beams. Two scans are acquired—either (1) back-to-back sequentially in time where the scans require two rotations around the subject, or (2) interleaved as a function of the rotation angle requiring one rotation around the subject, in which the tube operates at, for instance, 80 kVp and 160 kVp potentials. Special filters may be placed between the x-ray source and the detector such that different detector rows collect projections of different x-ray energy spectra. The special filters that shape the x-ray spectrum may be used for two scans that are acquired either back to back or interleaved. Energy sensitive detectors may be used such that each x-ray photon reaching the detector is recorded with its photon energy.

Techniques to obtain the measurements comprise: (1) scan with two distinctive energy spectra, and (2) detect photon energy according to energy deposition in the detector. EDCT/MECT/DE-CT provides energy discrimination and material characterization. For example, in the absence of object scatter, the system derives the behavior at a different energy based on the signal from two regions of photon energy in the spectrum: the low-energy and the high-energy portions of the incident x-ray spectrum. In a given energy region of medical CT, two physical processes dominate the x-ray attenuation: (1) Compton scatter and the (2) photoelectric effect. The detected signals from two energy regions provide sufficient information to resolve the energy dependence of the material being imaged. Furthermore, detected signals from the two energy regions provide sufficient information to determine the relative composition of an object composed of two hypothetical materials.

In EDCT/MECT/DE-CT, two or more sets of projection data are typically obtained for the imaged object at different tube peak kilovoltage (kVp) levels, which change the peak and spectrum of energy of the incident photons comprising the emitted x-ray beams or, alternatively, at a single tube peak kilovoltage (kVp) level or spectrum with an energy resolving detector of the detector array 18. The acquired sets of projection data may be used for basis material decomposition (BMD). During BMD, the measured projections are converted to a set of density line-integral projections. The density line-integral projections may be reconstructed to form a density map or image of each respective basis material, such as bone, soft tissue, and/or contrast agent maps. The density maps or images may be, in turn, associated to form a volume rendering of the basis material, for example, bone, soft tissue, and/or contrast agent, in the imaged volume.

Once reconstructed, the basis material image produced by the CT system 10 reveals internal features of the patient 22, expressed in the densities of the two basis materials. The density image may be displayed to show these features. In traditional approaches to diagnosis of medical conditions, such as disease states, and more generally of medical events, a radiologist or physician would consider a hard copy or display of the density image to discern characteristic features of interest. Such features might include lesions, sizes and shapes of particular anatomies or organs, and other features that would be discemable in the image based upon the skill and knowledge of the individual practitioner.

In addition to a CT number or Hounsfield value, an energy selective CT system can provide additional information related to a material's atomic number and density. This information may be particularly useful for a number of medical clinical applications, where the CT number of different materials may be similar but the atomic number may be quite different. For example, calcified plaque and iodine-contrast enhanced blood may be located together in coronary arteries or other vessels. As will be appreciated by those skilled in the art, calcified plaque and iodine-contrast enhanced blood are known to have distinctly different atomic numbers, but at certain densities these two materials are indistinguishable by CT number alone.

A decomposition algorithm is employable to generate atomic number and density information from energy sensitive x-ray measurements. Multiple energy techniques comprise dual energy, photon counting energy discrimination, dual layered scintillation and/or one or more other techniques designed to measure x-ray attenuation in two or more distinct energy ranges. As an example, a compound or mixture of materials measured with a multiple energy technique may be represented as a hypothetical material having the same x-ray energy attenuation characteristics. This hypothetical material can be assigned an effective atomic number Z. Unlike the atomic number of an element, effective atomic number of a compound is defined by the x-ray attenuation characteristics, and it need not be an integer. This effective Z representation property stems from a well-known fact that x-ray attenuation in the energy range useful for diagnostic x-ray imaging is strongly related to the electron density of compounds, which is also related to the atomic number of materials.

A conventional BMD algorithm is based on the concept that, in an energy region for CT scanning such as, for instance, in a medical patient, the x-ray attenuation of any given material can be represented by a proper density mix of two materials with distinct x-ray attenuation properties, referred to as the basis materials. The BMD algorithm computes two CT images that represent the equivalent density of one of the basis materials based on the measured projections at high and low x-ray photon energy spectra, respectively. Because of the strong energy dependence of x-ray attenuation coefficients and the polychromatic nature of the x-ray spectrum, conventional CT images typically contain beam hardening artifacts, except in a given material, typically water, used to calibrate the system. However, since a material density is independent of x-ray photon energy, beam-hardening artifacts can be greatly reduced or eliminated.

Classic approaches to EDCT recognize that the incident spectrum (in the absence of significant K-edges) can be expressed as the source spectrum attenuated through two path-lengths. Alternately, the attenuation can be modeled as components due to Compton scattering and photoelectric absorption. In either case, the classic approaches use two measurements for each ray to set up a system of two equations in two unknowns. In a monoenergetic case, the equations simplify to a system of two linear equations in two unknowns, which are solvable provided the determinant of the matrix is non-zero.

In a polychromatic case, the equations result in a non-linear relationship between the pathlengths of the two materials and the p-values (log-normalized intensity). The solution of these non-linear equations may be found, for example, using Newton's method for each pair of input sinogram values. An alternate approach may fit a polynomial to the inverse relationship between the measured p-values and the desired ones. The coefficients of this polynomial are determined by simple regression techniques once the space is suitably sampled.

In these approaches, the system of equations is critically determined (i.e., an equal number of equations and unknowns). As would be expected, though, image quality may be improved by acquiring more data. However, an excess of data will result in an overdetermined problem, which, if solved using one of the methods described above, will not result in full use of all the data available.

Therefore, it would be desirable to have a system and method to generate and directly solve an overdetermined set of CT measurements having energy diversity to provide an optimized, stable solution.

### BRIEF DESCRIPTION OF THE INVENTION

The present invention is directed to a system and method for directly solving an overdetermined set of energy diverse CT measurements that overcome the aforementioned drawbacks.

An energy discriminating CT detector capable of photon counting is disclosed. The CT detector supports not only x-ray photon counting, but energy measurement or tagging as well. The present invention supports the acquisition of both anatomical detail as well as tissue characterization information. These detectors support the acquisition of tissue discriminatory data and therefore provide diagnostic information that is indicative of disease or other pathologies. This detector can also be used to detect, measure, and characterize materials that may be injected into the subject such as contrast agents and other specialized materials.

According to an aspect of the present invention, a diagnostic imaging system includes an x-ray source that emits a beam of x-rays toward an object to be imaged, a detector that receives x-rays emitted by the x-ray source and attenuated by the object, and a data acquisition system (DAS) operably connected to the detector. A computer is operably connected to the DAS and programmed to obtain a number of measurements of energy-sensitive CT measurements in excess of a number of materials to be resolved, decompose the number of measurements into individual materials as an overdetermined system of equations, and generate an image of the individual materials based on the decomposition.

According to another aspect of the present invention, a method of diagnostic imaging includes acquiring a number of projections of energy sensitive CT data in excess of a number of basis functions to be resolved, decomposing the projections into equivalent path lengths through multiple basis functions as an overdetermined system of equations, and reconstructing each projection to get quantitative density information in the image domain.

According to yet another aspect of the present invention, a computer readable storage medium includes instructions stored thereon that, when executed by a processor, causes the computer to acquire a set of x-ray projection measurements of energy sensitive CT data as a series of line integrals, and decompose the line integrals into equivalent path lengths through multiple materials, wherein the number of measurements exceeds the number of materials, and an overdetermined set of equations and unknowns are solved simultaneously to minimize the residual error therein.

Various other features and advantages of the present invention will be made apparent from the following detailed description and the drawings.

### BRIEF DESCRIPTION OF THE DRAWINGS

The drawings illustrate one preferred embodiment presently contemplated for carrying out the invention.

In the drawings:

FIG. 1 is a pictorial view of a CT imaging system.

FIG. 2 is a block schematic diagram of the system illustrated in FIG. 1.

FIG. 3 is a perspective view of one embodiment of a CT system detector array.

FIG. 4 is a perspective view of one embodiment of a CT detector.

FIG. 5 is a cross-sectional view of one embodiment of a portion of a direct conversion detector.

FIG. 6 is a pictorial view of a CT system for use with a non-invasive package inspection system.

### DETAILED DESCRIPTION OF THE PREFERRED EMBODIMENT

Diagnostics devices comprise x-ray systems, magnetic resonance (MR) systems, ultrasound systems, computed tomography (CT) systems, positron emission tomography (PET) systems, ultrasound, nuclear medicine, and other types of imaging systems. Applications of x-ray sources comprise imaging, medical, security, and industrial inspection applications. However, it will be appreciated by those skilled in the art that an implementation is applicable for use with single-slice or other multi-slice configurations. Moreover, an implementation is employable for the detection and conversion of x-rays typically ranging from approximately 60-160 kV. However, one skilled in the art will further appreciate that an implementation is employable for the detection and conversion of other high frequency electromagnetic energy, such high-energy photons in excess of 160 kV. An implementation is employable with a “third generation” CT scanner and/or other CT systems.

The operating environment of the present invention is described with respect to a sixty-four-slice computed tomography (CT) system. However, it will be appreciated by those skilled in the art that the present invention is equally applicable for use with other multi-slice configurations. Moreover, the present invention will be described with respect to the detection and conversion of x-rays. However, one skilled in the art will further appreciate that the present invention is equally applicable for the detection and conversion of other high frequency electromagnetic energy. The present invention will be described with respect to a “third generation” CT scanner, but is equally applicable with other CT systems.

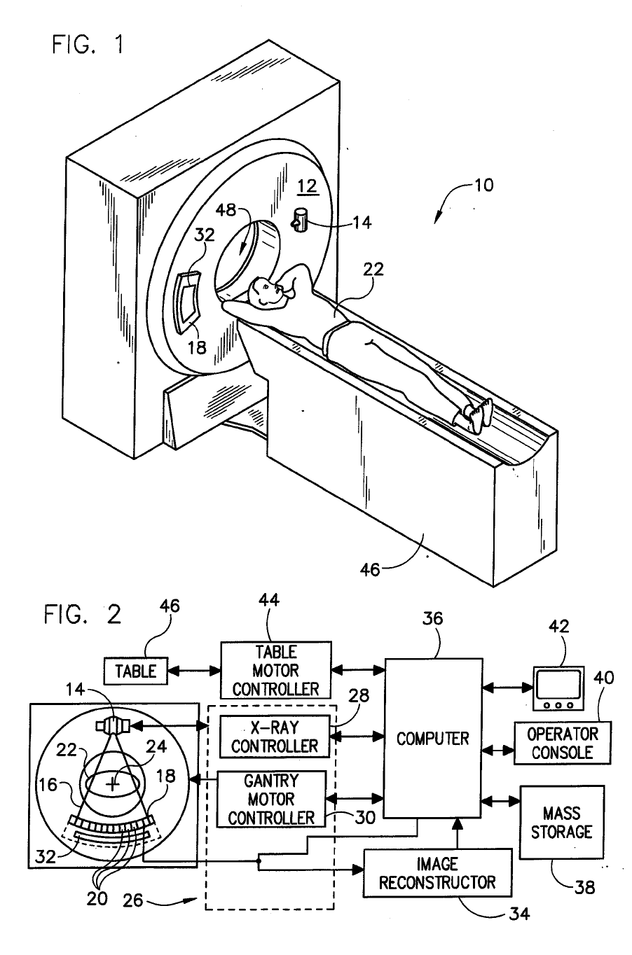

Referring to FIG. 1, a computed tomography (CT) imaging system 10 is shown as including a gantry 12 representative of a “third generation” CT scanner. Gantry 12 has an x-ray source 14 that projects a beam of x-rays 16 toward a detector assembly or collimator 18 on the opposite side of the gantry 12. Referring now to FIG. 2, detector assembly 18 is formed by a plurality of detectors 20 and data acquisition systems (DAS) 32. The plurality of detectors 20 sense the projected x-rays that pass through a medical patient 22, and DAS 32 converts the data to digital signals for subsequent processing. Each detector 20 produces an analog electrical signal that represents the intensity of an impinging x-ray beam and hence the attenuated beam as it passes through the patient 22. During a scan to acquire x-ray projection data, gantry 12 and the components mounted thereon rotate about a center of rotation 24.

Rotation of gantry 12 and the operation of x-ray source 14 are governed by a control mechanism 26 of CT system 10. Control mechanism 26 includes an x-ray controller 28 that provides power and timing signals to an x-ray source 14 and a gantry motor controller 30 that controls the rotational speed and position of gantry 12. An image reconstructor 34 receives sampled and digitized x-ray data from DAS 32 and performs high speed reconstruction. The reconstructed image is applied as an input to a computer 36 which stores the image in a mass storage device 38.

Computer 36 also receives commands and scanning parameters from an operator via console 40 that has some form of operator interface, such as a keyboard, mouse, voice activated controller, or any other suitable input apparatus. An associated display 42 allows the operator to observe the reconstructed image and other data from computer 36. The operator supplied commands and parameters are used by computer 36 to provide control signals and information to DAS 32, x-ray controller 28 and gantry motor controller 30. In addition, computer 36 operates a table motor controller 44 which controls a motorized table 46 to position patient 22 and gantry 12. Particularly, table 46 moves patients 22 through a gantry opening 48 of FIG. 1 in whole or in part.

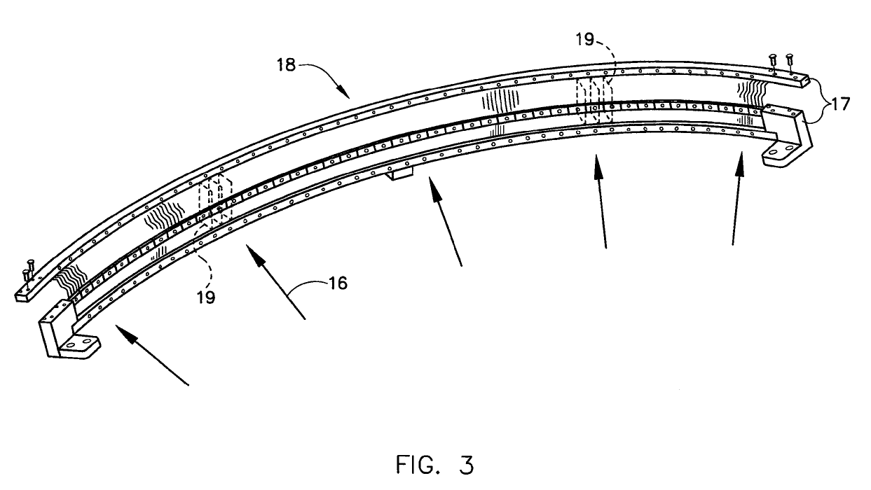

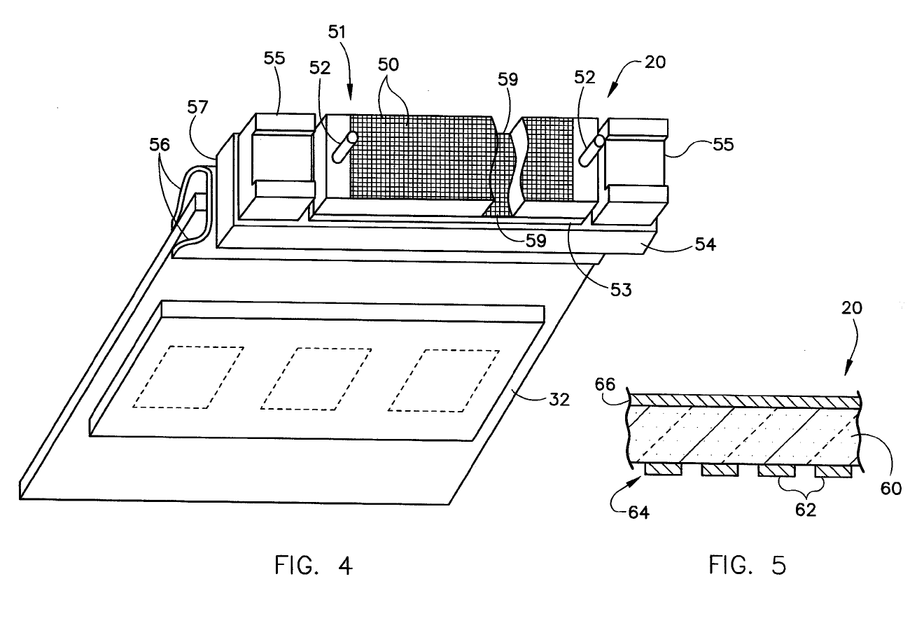

As shown in FIG. 3, detector assembly 18 includes rails 17 having collimating blades or plates 19 placed therebetween. Plates 19 are positioned to collimate x-rays 16 before such beams impinge upon, for instance, detector 20 of FIG. 4 positioned on detector assembly 18. In one embodiment, detector assembly 18 includes 57 detectors 20, each detector 20 having an array size of 64×16 of pixel elements 50. As a result, detector assembly 18 has 64 rows and 912 columns (16×57 detectors) which allows 64 simultaneous slices of data to be collected with each rotation of gantry 12.

Referring to FIG. 4, detector 20 includes DAS 32, with each detector 20 including a number of detector elements 50 arranged in pack 51. Detectors 20 include pins 52 positioned within pack 51 relative to detector elements 50. Pack 51 is positioned on a backlit diode array 53 having a plurality of diodes 59. Backlit diode array 53 is in turn positioned on multi-layer substrate 54. Spacers 55 are positioned on multi-layer substrate 54. Detector elements 50 are optically coupled to backlit diode array 53, and backlit diode array 53 is in turn electrically coupled to multi-layer substrate 54. Flex circuits 56 are attached to face 57 of multi-layer substrate 54 and to DAS 32. Detectors 20 are positioned within detector assembly 18 by use of pins 52.

In the operation of one embodiment, x-rays impinging within detector elements 50 generate photons which traverse pack 51, thereby generating an analog signal which is detected on a diode within backlit diode array 53. The analog signal generated is carried through multi-layer substrate 54, through flex circuits 56, to DAS 32 wherein the analog signal is converted to a digital signal.

As described above, each detector 20 may be designed to directly convert radiographic energy to electrical signals containing energy discriminatory or photon count data. In a preferred embodiment, each detector 20 includes a semiconductor layer fabricated from CZT. Each detector 20 also includes a plurality of metallized anodes attached to the semiconductor layer. As will be described, such detectors 20 may include an electrical circuit having multiple comparators thereon which may reduce statistical error due to pileup of multiple energy events.

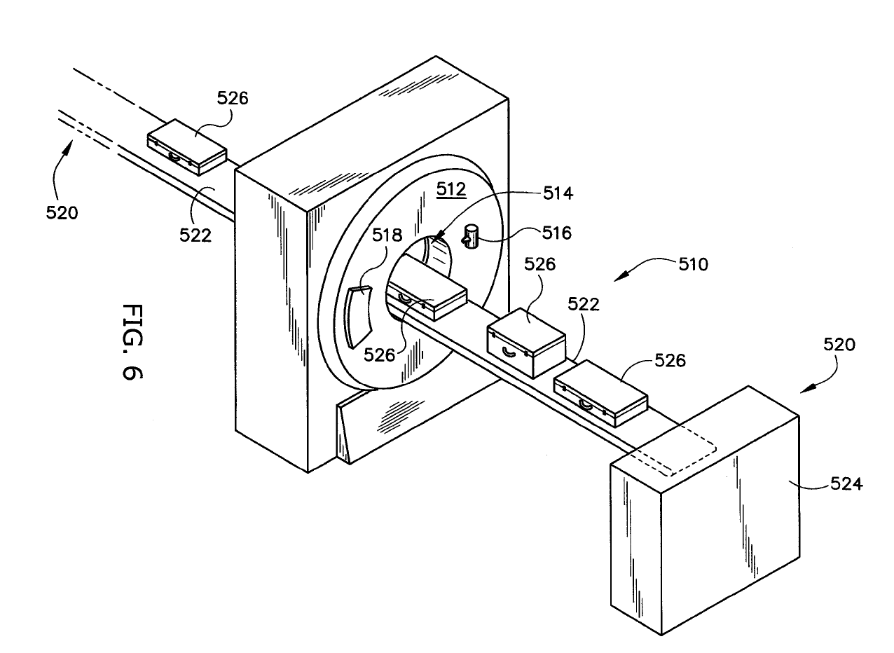

Referring now to FIG. 5, a portion of a CZT or direct conversion detector in accordance with one embodiment of the present invention is shown. Detector 20 is defined by a semiconductor layer 60 having a number of electronically pixelated structures or pixels to define a number of detector elements, anodes, or contacts 62. This electronic pixelation is accomplished by applying a 2D array 64 of electrical contacts 62 onto a layer 60 of direct conversion material.

Detector 20 includes a contiguous high-voltage electrode 66 attached to semiconductor layer 60. The high-voltage electrode 66 is connected to a power supply (not shown) and it is designed to power the semiconductor layer 60 during the x-ray detection process. One skilled in the art will appreciate that the high-voltage layer 66 should be relatively thin so as to reduce the x-ray absorption characteristics and, in a preferred embodiment, is a few hundred angstroms in thickness. In a preferred embodiment, the high-voltage electrode 66 may be affixed to the semiconductor layer 60 through a metallization process. X-ray photons that impinge upon the semiconductor layer 60 will generate an electrical charge therein, which is collected in one or more of the electrical contacts 62, and which may be read out to the DAS 32 of FIG. 2. The amplitude of the charge collected is indicative of the energy of the photon that created the charge.

Referring back to FIGS. 1 and 2, a typical decomposition algorithm is discussed. An image or slice is computed which may incorporate, in certain modes, less or more than 360 degrees of projection data to formulate an image. The image may be collimated to desired dimensions using tungsten blades in front of the x-ray source and different detector apertures. A collimator typically defines the size and shape of the beam of x-rays 16 that emerges from the x-ray source 14, and a bowtie filter may be included in the system 10 to further control the dose to the patient 22. A typical bowtie filter attenuates the beam of x-rays 16 to accommodate the body part being imaged, such as head or torso, such that, in general, less attenuation is provided for x-rays passing through or near an isocenter of the patient 22. The bowtie filter shapes the x-ray intensity during imaging in accordance with the region-of-interest (ROI), field of view (FOV), and/or target region of the patient 22 being imaged.

As the x-ray source 14 and the detector array 18 rotate, the detector array 18 collects data of the attenuated x-ray beams. The data collected by the detector array 18 undergoes pre-processing and calibration to condition the data to represent line integrals of the attenuation coefficients of the scanned object or the patient 22. The processed data are commonly called projections.

According to an embodiment of the present invention, a direct solution may be obtained for an overdetermined system of equations, taking advantage of excess of measurements to reduce residual error, yet avoiding a computationally demanding solution. In general, for a system of N components and M measurements, the overdetermined problem is set up when M>N. The generic intensity measurement equation is:

$${I}_{m} = \int {S}_{m} ( E ) exp ( - {\sum }_{n} {μ}_{n} ( E ) \int {ρ}_{n} d l ) d E , \qquad ( Eq . 1 )$$

wherein Iₘ represents the m-th intensity measurement for a given ray and where Sₘ(E) represents the spectral dependence of the measurement and is the product of the source spectrum, the detection spectrum, and the energy weighting function (if present). The quantity μₙ(E) is the mass attenuation coefficient for the n-th material, and ρₙ is the spatial density distribution for the n-th material. For brevity, qₙ will be denoted as ∫ηₙdl, which is the desired line-integral to be obtained for each component nε{1, . . . , N}. Intensities may be considered in terms of p-values, obtained by:

$${p}_{m} = - log \frac{\int {S}_{m} ( E ) exp ( - {\sum }_{n} {μ}_{n} ( E ) \int {ρ}_{n} d l ) d E}{\int {S}_{m} ( E ) d E} . \qquad ( Eqn . 2 )$$

For the special case of monoenergetic spectra, i.e., where Sₘ(E)=δ(E−Eₘ), and through simplification via the sifting theorem, substitution of Sₘ(E) into Eqn. 2 yields

$${p}_{m} = {\sum }_{n} {μ}_{n} ( {E}_{m} ) {q}_{n} ,$$

which is a linear system of equations having M equations and N unknowns that can be determined by standard linear techniques.

Generic approaches to solving Eqn. 2 for the polychromatic case are presented according to embodiments of the present invention. A solution wherein a polynomial expansion is used to replace the forward model may be used. The same can be done in a more general sense according to an embodiment of the present invention. In the more general case, the polynomial depends on the p-values for the M components. Accordingly, a generic polynomial of the form

$${p}_{m} = {\sum }_{k} {b}_{m , k} {\prod }_{n}^{ } {q}_{n}^{{c}_{k , n}} \qquad ( Eqn . 3 )$$

can be found (as c_{k,n} are known and fixed) by standard regression techniques where the coefficients b_{m,k} are the unknowns in a set of linear equations. The regression involves appropriate sampling of the path length combinations of the two materials (or attenuation effects) of interest. For each combination, the various qₙ are then given. Using Eqn. (2), the actual p values can be obtained, and these form the left hand side of Eqn. (3). A standard regression technique such as a minimum least squares method can then be used to find the coefficients b_{m,k}.

However, because of noise in the measurements, a consistent solution will most likely not exist that satisfies all of the equations, and the problem may be recast as a nonlinear weighted least squares problem, wherein:

$${q}^{*} = arg {min}_{q} {\sum }_{m} {{w}_{m} ( {p}_{m} - {F}_{m} ( q ) )}^{2} \qquad ( Eqn . 4 )$$

and wherein

$${F}_{m} ( q ) = - log \frac{\int {S}_{m} ( E ) exp ( - {\sum }_{n} {μ}_{n} ( E ) {q}_{n} ) d E}{\int {S}_{m} ( E ) d E} . \qquad ( Eqn . 4 a )$$

The use of Fₘ(q) as defined in equation (4a) may be cost prohibitive at run time. Instead, Fₘ(q) can be replaced with the polynomial expansion of Eqn. 4a. Note that in the 2-materials and 2-energies case, the determinant of the decomposition matrix was required to be nonzero for the problem to have a unique and stable solution. This result may be extended on the Jacobian of the transformation. The Jacobian must now be a full column rank for the problem to have a unique and stable solution. Note also, that because the problem is now overdetermined (there are more measurements than unknowns), we have the option of trading off the relative contributions of those measurements when formulating an answer. This trade off takes the form of weight factors in Eqn. 4, which are typically chosen to capture the relative statistical confidence in the measurements. In particular, measurements having a high photon count (thus a low uncertainty) will have a larger impact on the solution than measurements having a low photon count. Thus, correlations between the measurements may be taken via the weight matrix, which should model the joint covariance of the measurements.

While the embodiment of the present invention described above includes the ability to model the statistical confidence in the measurements at runtime, it still requires the solution of a nonlinear least squares problem for every measured ray in the sinogram.

Accordingly, another direct method of solution is described according to another embodiment of the present invention. Assuming a polynomial of the form:

$${q}_{n} = {\sum }_{k} {a}_{n , k} {\prod }_{m}^{ } {p}_{m}^{{d}_{k , m}} , \qquad ( Eqn . 5 )$$

where a_{n,k} is a vector having unknown coefficients, and d_{k,m} are known power terms in the polynomial expansion, this equation can be sampled at various values of qₙ, effectively computing pₘ for various path lengths through the N materials. Each one of these samples yields a linear relationship between the polynomial coefficients a_{n,k} and the desired output path length qₙ. This relationship can be expressed as a linear system of equations:

Pa=b (Eqn. 6),

wherein the columns of P correspond to different terms from the right hand side of Equation 5, and the rows P correspond to different path length combinations (i.e. for choices of the vector q). The left hand side a is a vector that includes the components of a_{n,k} ordered to match the column ordering of the power terms. The right hand side b is a vector that includes the desired output path lengths for each choice of input path lengths (i.e. b=[qₙ¹qₙ² . . . qₙ^{L}]) where L is the number of samples used to discretize the equations. Several sets of linear equations are solved in the form of Eqn. 6, one for each desired output for N total, but the computation can be solved prior to runtime thereby providing the vectors aₙ that can be used at runtime. Accordingly, at runtime, Eqn. 5 can then be used to compute the decomposition, which, as stated earlier, results in an overdetermined problem that can be solved using conventional least squares methods.

Because the generic solution is precomputed, prior to runtime, a large relative uncertainty of the measurements pᵢn occurs in the coefficients aᵢⱼ, but one skilled in the art would recognize that the uncertainty on the right hand side of Eqn. 6 is zero because the desired path length is as specified when calculating the forward model. Thus, the measurements appear as power terms in the elements of P, and the uncertainty of the measurement coefficients may be captured when solving for a.

According to another embodiment of the present invention, the error vector e may be defined as:

e=(P+Σ)a−b (Eqn. 7),

where Σ is a matrix-valued random variable representing noise of the elements of P. The vector a is unknown, so the error is parametrized in a and, according to this embodiment, a minimum mean squared solution (MMSE) may be determined for a.

More specifically:

aₘₘₛₑ*=arg minₐE{e^{T}e} (Eqn. 8),

which, when expanded and simplified, yields the following equations:

aₘₘₛₑ*=arg minₐE{[(P+Σ)a−b]^{T}[(P+Σ)a−b]} (Eqn. 9);

aₘₘₛₑ*=arg minₐE{a^{T}[(P+Σ)^{T}(P+Σ)a−2a^{T}P^{T}b−2a^{T}Σ^{T}b+b^{T}b]} (Eqn. 10);

aₘₘₛₑ*=arg minₐa^{T}(P^{T}P+2P^{T} Σ̅+X)a−2a^{T}(P^{T}b− Σ̅^{T}b)+b^{T}b (Eqn. 11);

aₘₘₛₑ*=(P^{T}P+2P^{T} Σ̅+X)⁻¹(P^{T}b− Σ̅^{T}b)^{T} (Eqn. 12),

where X is the autocorrelation of the error matrix, or

X=E{X^{T}X} (Eqn. 13).

One skilled in the art would recognize that the decomposition and the resulting estimator will likely be biased. However, as the error matrix Σ goes to 0, the solution converges to a standard, unbiased least squares solution, as the equations are asymptotically consistent.

One skilled in the art would recognize that it may be desirable to have an unbiased decomposition and a higher mean squared error as a result. Thus, the decomposition may be solved for an unbiased estimator having a minimum variance according to another embodiment of the present invention. The minimum variance unbiased estimator (MVUE) may be found by forcing the error vector to zero. That is:

E{e}=0=(P+ Σ̅)a−b (Eqn. 14).

In the mean squared error expression above, we can write and simplify:

aₘₘₛₑ*=arg _{a,(P+ Σ̅)a=b}a^{T}(P^{T}P+2P^{T} Σ̅+X)a−2a^{T}(P^{T}b− Σ̅^{T}b)+b^{T}b ; (Eqn. 15),

aₘₘₛₑ*=arg _{a,(P+ Σ̅)a=b}a^{T}(P^{T}P+2P^{T} Σ̅+X)a−2[(P− Σ̅a]^{T}b+b^{T}b (Eqn. 16),

aₘₘₛₑ*=arg _{a,(P+ Σ̅)a=b}a^{T}(P^{T}P+2P^{T} Σ̅+X)a−b^{T}b (Eqn. 17),

which is a linearly constrained quadratic minimization problem to which one skilled in the art would recognize that the solution for this may be found using standard techniques, resulting in a minimum variance, unbiased estimator. One skilled in the art would recognize that the mean constraint, Eqn. 14, may force a unique solution (where requiring the estimator to be unbiased may lead to a single solution or even to no solution) and may result in a solution less desirable than a solution allowing for bias in the estimator. Thus, in practice, the MMSE solution may be preferred to the MVUE solution. Additionally, Eqn. 14 may have no solution even if Σ̅=0, in which case estimators that are unbiased in the least squares sense (i.e., the mean error vector minimized the least squares residual) may then be desirable.

Regarding the calculations of Σ̅ and X, the elements of P are coupled through a power series expansion of the measured p values. Therefore, one skilled in the art would recognize that the matrices Σ̅ and X can readily be calculated through knowledge of the uncertainties on the pᵢₙ. Note also, that these calculations can be performed ahead of time, and stored for rapid retrieval on the system. For example, if large path lengths imply a degree of photon starvation (and hence a low statistical confidence in some of the measurements), a model can predict this phenomenon. This prediction can then be used to construct the Σ̅ and X matrices. A particular example of this phenomenon in photon counting detectors is the effects of pileup at high fluxes, and photon starvation at low fluxes.

Referring now to FIG. 6, package/baggage inspection system 510 includes a rotatable gantry 512 having an opening 514 therein through which packages or pieces of baggage may pass. The rotatable gantry 512 houses an x-ray and/or high frequency electromagnetic energy source 516 as well as a detector assembly 518 having scintillator arrays comprised of scintillator cells. A conveyor system 520 is also provided and includes a conveyor belt 522 supported by structure 524 to automatically and continuously pass packages or baggage pieces 526 through opening 514 to be scanned. Objects 526 are fed through opening 514 by conveyor belt 522, imaging data is then acquired, and the conveyor belt 522 removes the packages 526 from opening 514 in a controlled and continuous manner. As a result, postal inspectors, baggage handlers, and other security personnel may non-invasively inspect the contents of packages 526 for explosives, knives, guns, contraband, etc. An exemplary implementation can aid in the development of automatic inspection techniques, such as explosive detection in luggage.

An implementation of the system 10 and/or 510 in an example comprises a plurality of components such as one or more of electronic components, hardware components, and/or computer software components. A number of such components can be combined or divided in an implementation of the system 10 and/or 510. An exemplary component of an implementation of the system 10 and/or 510 employs and/or comprises a set and/or series of computer instructions written in or implemented with any of a number of programming languages, as will be appreciated by those skilled in the art. An implementation of the system 10 and/or 510 in an example comprises any (e.g., horizontal, oblique, or vertical) orientation, with the description and figures herein illustrating an exemplary orientation of an implementation of the system 10 and/or 510, for explanatory purposes.

An implementation of the system 10 and/or the system 510 in an example employs one or more computer readable signal bearing media. A computer-readable signal-bearing medium in an example stores software, firmware and/or assembly language for performing one or more portions of one or more implementations. An example of a computer-readable signal bearing medium for an implementation of the system 10 and/or the system 510 comprises the recordable data storage medium of the image reconstructor 34, and/or the mass storage device 38 of the computer 36. A computer-readable signal-bearing medium for an implementation of the system 10 and/or the system 510 in an example comprises one or more of a magnetic, electrical, optical, biological, and/or atomic data storage medium. For example, an implementation of the computer-readable signal-bearing medium comprises floppy disks, magnetic tapes, CD-ROMs, DVD-ROMs, hard disk drives, and/or electronic memory. In another example, an implementation of the computer-readable signal-bearing medium comprises a modulated carrier signal transmitted over a network comprising or coupled with an implementation of the system 10 and/or the system 510, for instance, one or more of a telephone network, a local area network (“LAN”), a wide area network (“WAN”), the Internet, and/or a wireless network.

Therefore, according to an embodiment of the present invention, a diagnostic imaging system includes an x-ray source that emits a beam of x-rays toward an object to be imaged, a detector that receives x-rays emitted by the x-ray source and attenuated by the object, and a data acquisition system (DAS) operably connected to the detector. A computer is operably connected to the DAS and programmed to obtain a number of measurements of energy-sensitive CT measurements in excess of a number of materials to be resolved, decompose the number of measurements into individual materials as an overdetermined system of equations, and generate an image of the individual materials based on the decomposition.

According to another embodiment of the present invention, a method of diagnostic imaging includes acquiring a number of projections of energy sensitive CT data in excess of a number of basis functions to be resolved, decomposing the projections into equivalent path lengths through multiple basis functions as an overdetermined system of equations, and reconstructing each projection to get quantitative density information in the image domain.

According to yet another embodiment of the present invention, a computer readable storage medium includes instructions stored thereon that, when executed by a processor, causes the computer to acquire a set of x-ray projection measurements of energy sensitive CT data as a series of line integrals, and decompose the line integrals into equivalent path lengths through multiple materials, wherein the number of measurements exceeds the number of materials, and an overdetermined set of equations and unknowns are solved simultaneously to minimize the residual error therein.

The present invention has been described in terms of the preferred embodiment, and it is recognized that equivalents, alternatives, and modifications, aside from those expressly stated, are possible and within the scope of the appending claims.

What is claimed is:

1. A diagnostic imaging system comprising:
an x-ray source that emits a beam of x-rays toward an object to be imaged;
a detector that receives x-rays emitted by the x-ray source and attenuated by the object;
a data acquisition system (DAS) operably connected to the detector; and
a computer operably connected to the DAS and programmed to:
obtain a number of measurements of energy-sensitive CT measurements in excess of a number of materials to be resolved;
decompose the number of measurements into individual materials as an overdetermined system of equations; and
generate an image of the individual materials based on the decomposition.

2. The imaging system of claim 1 wherein the computer is further programmed to generate at least one sinogram for each material of the number of materials to be resolved.

3. The imaging system of claim 1 wherein the computer, in being programmed to decompose the number of measurements, is programmed to decompose the measurements in a non-linear weighted least squares fashion.

4. The imaging system of claim 3 wherein the computer, in being programmed to decompose the number of measurements, is programmed to decompose the measurements using substantially every ray in a sinogram.

5. The imaging system of claim 1 wherein the computer is further programmed to solve a linear system of equations in the decomposition resulting in a number of vectors and, in being programmed to decompose the number of measurements, use the resulting vectors at runtime to decompose the measurements.

6. The imaging system of claim 5 further comprising an error vector e that is parametrized by an unknown vector a, and solved for using a minimum mean squared error (MMSE) of the unknown vector a.

7. The imaging system of claim 5 wherein an unbiased decomposition is obtained having a minimized variance unbiased estimator (MVUE) by using a linearly constrained quadratic minimization technique.

8. A method of diagnostic imaging comprising:
acquiring a number of projections of energy sensitive CT data in excess of a number of basis functions to be resolved;
decomposing the projections into equivalent path lengths through multiple basis functions as an overdetermined system of equations; and
reconstructing each projection to get quantitative density information in the image domain.

9. The method of diagnostic imaging of claim 8 further comprising generating a sinogram for each basis function.

10. The method of claim 8 further comprising decomposing the projections as a non-linear weighted least squares problem.

11. The method of claim 10 further comprising solving the non-linear weighted least squares problem using an iterative technique.

12. The method of claim 8 wherein the step of decomposing further comprises:
formulating the basis functions as polynomial functions;
obtaining a system of linear equations therefrom; and
solving the system using a least squares technique.

13. The method of claim 12 wherein the step of solving comprises:
generating an error vector e that is parametrized by an unknown vector a; and
using a minimum mean squared error (MMSE) of the unknown vector a.

14. The method of claim 12 further comprising obtaining an unbiased decomposition and having a minimized variance unbiased estimator (MVUE) by using a linearly constrained quadratic minimization technique.

15. A computer readable storage medium having stored thereon instructions that, when executed by a processor, cause a computer to:
acquire a set of x-ray projection measurements of energy sensitive CT data as a series of line integrals; and
decompose the line integrals into equivalent path lengths through multiple materials;
wherein the number of measurements exceeds the number of materials, and an overdetermined set of equations and unknowns are solved simultaneously to minimize the residual error therein.

16. The computer readable storage medium of claim 15 wherein the computer is further caused to generate at least one sinogram for each material of the number of materials to be resolved.

17. The computer readable storage medium of claim 15 wherein the computer is caused to decompose the line integrals as a nonlinear weighted least squares problem.

18. The computer readable storage medium of claim 17 wherein the computer is further caused to decompose the line integrals using substantially every ray in a sinogram.

19. The computer readable storage medium of claim 15 wherein the computer is further caused to solve a linear system of equations prior to data acquisition and use the resulting vectors at runtime to decompose the line integrals.

20. The computer readable storage medium of claim 19 wherein an error vector e that is parametrized by an unknown vector a, and solved for using a minimum mean squared error (MMSE) of the unknown vector a.

21. The computer readable storage medium of claim 19 wherein an unbiased decomposition is obtained having a minimized variance unbiased estimator (MVUE) by using a linearly constrained quadratic minimization technique.
